(19) United States
(12) Patent Application Publication
(10) Pub. No.: US 2005/0255064 A1
(43) Pub. Date: Nov. 17, 2005

(54) Antiperspirant composition

(76) Inventors: Stefan Bruening, Duesseldorf (DE); Achim Ansmann, Erkrath (DE); Helga Gondek, Duesseldorf (DE); Bettina Jackwerth, Langenfeld (DE)

Correspondence Address: COGNIS CORPORATION;PATENT DEPARTMENT, 300 BROOKSIDE AVENUE, AMBLER, PA, 19002, US

(21) Appl. No.: 10/498,773

(22) PCT Filed: Dec. 9, 2002
(86) PCT No.: PCT/EP02/13929

§ 371 Date: May 5, 2005

(30) Foreign Application Priority Data
Dec. 17, 2001 (DE) 101 02 049.7

(51) Int. Cl. A61K 7/34; A61K 7/36

(52) U.S. Cl. 424/66; 424/67

(57) ABSTRACT

An antiperspirant composition containing: (a) at least one dialkyl carbonate corresponding to formula I: 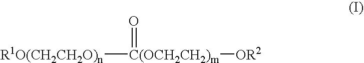
wherein R¹ and R² represent a linear or branched, alkyl and/or alkenyl, group containing from about 16 to 60 carbon atoms, and n and m, independently of one another, represent a number up to 100; (b) at least one antiperspirant component; (c) optionally, a wax component other than the dialkyl carbonate; and (d) optionally, an oil component, and wherein the composition contains less than about 5% by weight, based on the weight of the composition, of water.

### FIELD OF THE INVENTION

This invention relates to water-free antiperspirant compositions containing long-chain C₁₆₋₆₀ dialkyl carbonates and antiperspirant components.

### PRIOR ART

Antiperspirant compositions are known to experts in many different forms. The antiperspirant components normally used are astringent substances, for example aluminium and/or zirconium salts. The compositions are marketed as sprays, roll-on preparations, sticks or creams. Corresponding formulations are described by S. Plechner in Cosmetics: Science and Technology, “Antiperspirants and Deodorants” (Eds. M. S. Balsam and E. G. Sagarin, 1972), Vol. 2, pages 373-416, and in Cosmetics Science and Technology Series: “Antiperspirants and Deodorants” (Ed.: Karl Laden), 2nd Edition, pages 233-258 and 327-356.

In recent years, water-free cream and stick preparations have become increasingly popular on the cosmetics market. Fatty alcohols, such as cetearyl, stearyl and behenyl alcohol for example, and hydroxyfatty acids, for example 12-hydroxystearic acid, are often used as a so-called wax base in water-free antiperspirant sticks and so-called soft solid formulations. Corresponding stick preparations are disclosed, for example, in U.S. Pat. No. 4,822,603, U.S. Pat. No. 4,126,679 and EP 117 070.

Water-free stick preparations containing volatile silicone oils have the disadvantage that the dispersed active components lead to visible product residues on the skin and clothing. If pressure is applied during application, “oiling out” (syneresis) often occurs and reduces the cosmetic acceptance of the preparations among users. In addition, the fatty alcohol base leaves the skin with an unsatisfactory feeling and frequently causes dermal irritation, even if the other emollients are optimally selected.

The problem addressed by the present invention was to develop water-free antiperspirant formulations with improved sensory properties and dermatological compatibility which would lead to reduced residue formation (“white residue”) on skin and clothing. Another problem addressed by the invention was to improve the particle fineness and smoothness (structure) and also the long-term storage stability of the formulations.

### DESCRIPTION OF THE INVENTION

It has been found that long-chain C₁₆₋₆₀ dialkyl carbonates significantly improve the product properties of water-free antiperspirant formulations.

Accordingly, the present invention relates to water-free antiperspirant preparations containing
(a) at least one dialkyl carbonate corresponding to formula (I): 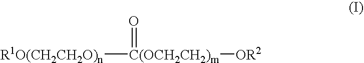
   in which R¹ and R² represent a linear or branched alkyl and/or alkenyl group containing 16 to 60 carbon atoms and n and m independently of one another stand for 0 or for numbers of 1 to 100, and
(b) at least one antiperspirant component.

Water-free compositions in the context of the present invention are compositions which contain less than 5% by weight of water (not including water of crystallization), preferably less than 2% by weight of water and more particularly less than 1% by weight of water. Residual water may emanate from the raw materials used and may therefore be unavoidable.

The C₁₆₋₆₀ dialkyl carbonates, either on their own or in combination with additional wax-like substances, have a strong consistency-imparting effect on various emollients and are therefore particularly suitable for use in antiperspirant soft solid and stick formulations. Besides the pure consistency-providing properties, extremely homogeneous inner structures and homogeneous, smooth surface structures are obtained in the formulations according to the invention. The wax-like C₁₆₋₆₀ dialkyl carbonates improve the overall sensory impression of the water-free antiperspirant formulation. Thus, greater smoothness and softness of the skin are obtained which leads to an improved care effect. The formation of residues on skin and clothing (“white residue”) is clearly reduced by the compositions according to the invention so that the products are distinguished by increased cosmetic acceptance among users. The present invention also relates to the use of C₁₆₋₆₀ dialkyl carbonates in antiperspirant formulations, more particularly in water-free antiperspirant formulations, more particularly for reducing residue formation.

These long-chain dialkyl carbonates may generally be used with considerable advantage for the formulation of stick compositions, for example in make-up sticks, in lipsticks, lip care sticks, sticks with insect repellent properties or the like.

Dialkyl Carbonates

Dialkyl carbonates which form component (a) are basically known substances. The use of short-chain dialkyl carbonates as liquid oil components in antiperspirant formulations is known, for example, from DE 100 02 643. The substances may be prepared in known manner by transesterification of dimethyl or diethyl carbonate with the corresponding hydroxy compounds; an overview of relevant processes can be found, for example, in Chem. Rev. 96, 951 (1996).

Examples of such dialkyl carbonates are the complete, symmetrical or nonsymmetrical transesterification products of dimethyl and/or diethyl carbonate with cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures of these alcohols which are obtained, for example, in the high-pressure hydrogenation of technical methyl esters based on fats and oils or aldehydes from Roelen's oxo synthesis and as monomer fraction in the dimerization of unsaturated fatty alcohols. Transesterification products of the lower carbonates with the alcohols mentioned in the form of their adducts with 1 to 100, preferably 2 to 50 and, more particularly, 5 to 20 mol ethylene oxide are also suitable.

Dialkyl carbonates of formula (I), which are particularly suitable for solving the problem addressed by the invention, are compounds of wax-like consistency which are solid at 23° C. Compounds in which R¹ and R² independently of one another represent a linear saturated alkyl group containing 18 to 30 carbon atoms and m and n have a value of 0 are particularly suitable. Symmetrical dialkyl carbonates where R¹ and R² are identical are preferred, dioctadecyl carbonate being most particularly preferred.

Antiperspirant Component

In principle, any astringent salts which reduce perspiration are suitable as the antiperspirant component (component b). These salts are preferably astringent aluminium compounds, aluminium zirconium compounds or zinc salts. Examples include aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Aluminium chlorohydrate, aluminium zirconium tetrachlorohydrate, aluminium zirconium pentachlorohydrate and complex compounds thereof are preferably used. The use of compounds commercially available as Locron® P and Rezal® 36 GP is particularly preferred.

The preparations according to the invention preferably contain (a) 0.1 to 40% by weight C₁₆₋₆₀ dialkyl carbonates and (b) 5 to 45% by weight of antiperspirants, more particularly (a) 1 to 30% by weight C₁₆₋₆₀ dialkyl carbonates and (b) 5 to 30% by weight antiperspirants and, in a particularly preferred embodiment, (a) 2 to 25% by weight C₁₆₋₆₀ dialkyl carbonates and (b) 15 to 30% by weight antiperspirants. After a penetration time of 5 seconds at 23° C., the preparations according to the invention preferably have a penetration depth of 1.0 to 40.0 mm (Penetrometer PNR 10 Petrotest; Petrotest Instruments GmbH & Bo. KG; microcone: 5.0 g; drop bar: 47.5 g; measuring temperature: 23° C.; measuring time: 5 seconds). The penetration depth is thus a measure of the “hardness” or consistency of the antiperspirant. The lower the depth of penetration, the “harder” the antiperspirant.

Waxes

In another preferred embodiment, the compositions according to the invention contain at least one other wax which contributes towards optimizing the sensory properties, the consistency and the stability of the sticks. According to the invention, this other wax (for a definition, see CD Römpp Chemie Lexikon—Version 1.0, Stuttgart/New York: Georg Thieme Verlag 1995) may be selected from any natural and synthetic substances of wax-like consistency. These include inter alia fats (triglycerides), mono- and diglycerides, waxes, fatty and wax alcohols, fatty acids, esters of fatty alcohols and also fatty acid amides or mixtures of these substances. They may be present in the compositions according to the invention in a total quantity of 0.1 to 40% by weight, preferably 1 to 30% by weight and more particularly 5 to 20% by weight.

Fats

Fats in the context of the invention are understood to be triacylglycerols, i.e. the triple esters of fatty acids with glycerol. Among the triacylglycerols, those which melt at 30 to 80° C. and more particularly at 40 to 60° C. are preferred. The triacylglycerols preferably contain saturated, unbranched and unsubstituted fatty acid components. They may also be mixed esters, i.e. triple esters of glycerol with various fatty acids. So-called hardened fats and oils obtained by partial hydrogenation may be used in accordance with the invention and are particularly suitable as consistency factors. Vegetable hardened fats and oils, for example hardened castor oil, peanut oil, soybean oil, colza oil, rapeseed oil, cottonseed oil, soybean oil, sunflower oil, palm oil, palm kernel oil, linseed oil, almond oil, corn oil, olive oil, sesame oil, cocoa butter and coconut fat, are preferred.

Suitable fats are inter alia the triple esters of glycerol with C₁₂₋₆₀ fatty acids and in particular C₁₂₋₃₆ fatty acids. These include hydrogenated castor oil, a triple ester of glycerol and a hydroxystearic acid which is marketed, for example, under the name of Cutina® HR. The use of hardened oils and fats, more particularly Cutina® HR, is preferred for the purposes of the invention. Glycerol tristearate, glycerol tribehenate (for example Syncrowax® HRC), glycerol tripalmitate or the triglyceride mixtures known under the name of Syncrowax® HGLC are also suitable.

Besides the triglycerides, other suitable lipid components are mono- and diglycerides and mixtures of glycerides. According to the invention, preferred glyceride mixtures include the products Novata® AB and Novata® B (mixture of C₁₂₋₁₈ mono-, di- and triglycerides) and Cutina® MD or Cutina® GMS (glyceryl stearate) marketed by Cognis Deutschland GmbH.

Mixed esters and mixtures of mono-, di- and triglycerides are particularly suitable for the purposes of the invention because they have a relatively low tendency towards crystallization and thus improve the performance of the composition according to the invention.

Fatty Alcohols and Fatty Acids

Fatty alcohols of wax-like consistency suitable for use in accordance with the invention include C₁₂₋₅₀ fatty alcohols, more particularly C₁₂₋₂₄ fatty alcohols obtained from natural fats, oils and waxes such as, for example, myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol and Guerbet alcohols. According to the invention, saturated, branched or unbranched fatty alcohols are preferred. Other suitable fatty alcohols are the fatty alcohol cuts obtained in the reduction of naturally occurring fats and oils such as, for example, bovine tallow, peanut oil, colza oil, cottonseed oil, soybean oil, sunflower oil, palm kernel oil, linseed oil, castor oil, corn oil, rapeseed oil, sesame oil, cocoa butter and coconut oil. However, synthetic alcohols, for example the linear, even-numbered fatty alcohols from Ziegler's synthesis (Alfols®) or the partly branched alcohols from the oxosynthesis (Dobanols®) may also be used. A preferred embodiment of the composition according to the invention contains at least one C₁₂₋₁₄ fatty alcohol or a combination of a C₁₂₋₂₄ fatty alcohol and a hardened triglyceride as another wax. The combination of stearyl alcohol and hydrogenated castor oil is particularly preferred. C₁₄₋₁₈ fatty alcohols marketed for example by Cognis Deutschland GmbH under the name of Lanette® 16 (C₁₆ alcohol), Lanette® 14 (C₁₄ alcohol), Lanette® O (C_{16/18} alcohol) and Lanette® 22 (C_{18/22} alcohol) are particularly suitable for the purposes of the invention. Fatty alcohols give the compositions a dryer feeling on the skin than triglycerides.

C₁₄₋₄₀ fatty acids or mixtures thereof may be used as additional wax-like lipid components. These include, for example, myristic, pentadecanoic, palmitic, margaric, stearic, nonadecanoic, arachic, behenic, lignoceric, cerotic, melissic, erucic and elaeostearic acid and substituted fatty acids such as, for example, 12-hydroxystearic acid, and the amides or monoethanolamides of the fatty acids. This list is meant to be purely exemplary without any limiting character. Among the fatty acids, 12-hydroxystearic acid is particularly preferred.

Waxes

Waxes suitable for use in accordance with the present invention are, for example, natural vegetable waxes, such as candelilla wax, carnauba wax, Japan wax, espartograss wax, cork wax, guaruma wax, rice oil wax, sugar cane wax, ouricury wax, montan wax, sunflower wax, fruit waxes, such as orange waxes, lemon waxes, grapefruit wax, bayberry wax, and animal waxes such as, for example, beeswax, shellac wax, spermaceti, wool wax and uropygial fat. According to the invention, it can be of advantage to use hydrogenated or hardened waxes. Natural waxes usable in accordance with the invention also include the mineral waxes, such as ceresine and ozocerite for example, or the petrochemical waxes, for example petrolatum, paraffin waxes and microwaxes. Other suitable wax components are chemically modified waxes, more particularly the hard waxes such as, for example, montan ester waxes, sasol waxes and hydrogenated jojoba waxes. Synthetic waxes usable in accordance with the invention include, for example, wax-like polyalkylene waxes and polyethylene glycol waxes.

The wax component may also be selected from the group of esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (for example 12-hydroxystearic acid) and saturated and/or unsaturated, branched and/or unbranched alcohols and also from the group of lactides of long-chain hydroxycarboxylic acids. Wax components such as these include, for example, C₁₆₋₄₀ alkyl stearates, C₂₀₋₄₀ alkyl stearates (for example Kesterwachs® K82H), C₂₀₋₄₀ dialkyl esters of dimer acids, C₁₈₋₃₈ alkyl hydroxystearoyl stearates or C₂₀₋₄₀ alkyl erucates. Other suitable wax components which may be used with advantage are C₃₀₋₅₀ alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate. Silicone waxes may also be used with advantage.

Oil Components

In another preferred embodiment, the composition according to the invention contains at least one oil component. In the context of the invention, oil components are substances or mixtures of substances which are liquid at 20° C. and immiscible with water at 25° C. Such substances include, for example, glycerides, hydrocarbons, silicone oils, ester oils, dialkyl(ene) ethers, dialkyl(ene) carbonates liquid at 20° C. or mixtures thereof. The oil components are present in the compositions according to the invention in total quantities of normally less than 95% by weight, preferably 10 to 85% by weight and more particularly 30 to 85% by weight, based on the composition as a whole.

Glycerides suitable as oil components in accordance with the invention include fatty acid esters of glycerol liquid at 20° C. which may be of natural (animal and vegetable) or synthetic origin. Glycerides are divided into mono-, di- and triglycerides. They are known substances which may be obtained by the relevant methods of preparative organic chemistry. Synthetic glycerides are normally mixtures of mono-, di and triglycerides which are obtained by transesterification of the corresponding triglycerides with glycerol or by selective esterification of fatty acids. Preferred fatty acids for the purposes of the invention are C₆₋₂₄ fatty acids and, among these, C₆₋₁₈ fatty acids and especially C₈₋₁₈ fatty acids. The fatty acids may be branched or unbranched, saturated or unsaturated. According to the invention, it is preferred to use glycerides of vegetable origin liquid at 20° C., more particularly cocoglycerides, a mixture of predominantly di- and triglycerides with C₈₋₁₈ fatty acids marketed under the name of Myritol® 331 by Cognis Deutschland GmbH. It is also preferred to use Myritol® 312 (C_{8/10} triglycerides), Cegesoft® PS 17, Cegesoft® GPO, Cegesoft® PFO and Cegesoft® PS 6.

A preferred embodiment of the invention contains at least one oil component selected from the group of dialkyl ethers liquid at 20° C. or dialkyl carbonates liquid at 20° C. or a combination of these substances. Oil components such as these provide the compositions with particularly good skin-care properties and a pleasantly dry feeling on the skin after application. The combination of a dialkyl ether liquid at 20° C. with cyclohexane derivatives is also preferred. A particularly preferred oil component combination for the compositions according to the invention contains Cetiol® OE and Cetiol® S.

Other suitable oil components are Guerbet alcohols liquid at 20° C. based on fatty alcohols containing 6 to 18 and preferably 8 to 10 carbon atoms, such as Eutanol® G for example. Liquid esters of linear, saturated or unsaturated C₆₋₂₂ fatty acids with linear or branched, saturated or unsaturated C₆₋₂₂ fatty alcohols or esters of branched C₆₋₁₃ carboxylic acids with linear or branched, saturated or unsaturated C₆₋₂₂ fatty alcohols, such as Cetiol® CC for example, may also be used as oil components in accordance with the invention.

Examples of wax esters liquid at 20° C. include the following typical representatives: decyl oleate (Cetiol® V), cococaprylate/caprate (Cetiol® SN), hexyl laurate (Cetiol® A), myristyl isostearate, myristyl oleate, cetyl isostearate, cetyl oleate, stearyl isostearate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate (Cetiol® DAB), oleyl behenate, oleyl erucate (Cetiol® J 600), behenyl isostearate, erucyl isostearate, erucyl oleate. Also suitable are esters of linear C₆₋₂₂ fatty acids with branched alcohols, more particularly 2-ethyl hexanol (Cetiol® 868), esters of branched C₆₋₂₂ fatty acids with linear alcohols, esters of C₁₈₋₃₈ alkylhydroxy-carboxylic acids with linear or branched C₆₋₂₂ fatty alcohols, esters of linear and/or branched fatty acids with polyhydric alcohols (for example propylene glycol, dimer diol or trimer triol) and/or Guerbet alcohols and esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms (for example Dioctyl Malate) or polyols containing 2 to 10 carbon atoms and 2 to 6 hydroxyl groups.

Other oil components suitable for use in accordance with the invention are natural and synthetic, aliphatic and/or naphthenic hydrocarbons liquid at 20° C., such as for example squalane, squalene, paraffin oils, isohexadecane, isoeicosane or polydecenes and dialkyl cyclohexanes (Cetiol®).

According to the invention, other suitable oil components are liquid silicone oils. The addition of silicone compounds imparts a particularly light feeling on the skin. The silicone compounds include, for example, dialkyl and alkylaryl siloxanes, such as for example cyclomethicone, dimethyl polysiloxane and methylphenyl polysiloxane and alkoxylated and quaternized analogs thereof. Suitable nonvolatile silicone oils, such as for example polyalkyl siloxanes, polyalkylaryl siloxanes and polyether siloxane copolymers are described in Cosmetics: Science and Technology, eds.: M. Balsam and E. Sagarin, Vol. 1,1972, pp. 27-104, in U.S. Pat. No. 4,202,879 and U.S. Pat. No. 5,069,897. A particularly preferred embodiment of the composition according to the invention additionally contains at least one oil component selected from the group of volatile silicone compounds. Volatile compounds in the context of the invention are compounds which volatilize at body temperature. Suitable volatile silicone oils, which may be linear, branched or cyclic, are described in Todd et al. “Volatile Silicone Fluids for Cosmetics”, Cosmetics and Toiletries, pp. 27-32 (1976). Silicone oils containing 3 to 7 and more particularly 4 to 6 silicon atoms are preferred for the purposes of the invention. Particularly preferred are cyclic polydimethylsiloxanes such as, for example, octamethyl cyclotetrasiloxane, decamethyl cyclopentasiloxane or dodecamethyl cyclohexasiloxane which are known as cyclomethicones. Sensorially, they contribute to a very dry skin feel. They are commercially obtainable from G. E. Silicones as Cyclomethicone D-4 and D-5, from Dow Corning Corp. as Dow Corning® 344, 345 and 244, 245, 246, from General Electric Co. as GE® 7207 and 7158. Of the linear volatile silicones, those containing 1 to 7 and preferably 2 to 3 silicon atoms are preferred. The volatile silicones are present in a quantity of 1 to 60% by weight, preferably in a quantity of 1 to 40% by weight and more particularly in a quantity of 10 to 35% by weight.

A particularly preferred embodiment of the composition according to the invention contains:
(a) 1 to 40% by weight of at least one C₁₆₋₆₀ dialkyl carbonate,
(b) 5 to 45% by weight of at least one antiperspirant component,
(c) 0.1 to 20% by weight of at least one liquid dialkyl ether and
(d) 10 to 60% by weight of at least one volatile silicone oil.
   Additional Deodorant Components

In order to achieve optimal overall performance, the compositions according to the invention may contain a number of other active substances and components which are listed in the following.

Esterase Inhibitors

Where perspiration is present in the axillary region, extracellular enzymes—esterases, preferably proteases and/or lipases—are formed by bacteria, cleave the esters present in the perspiration and thus release odor-forming substances. Substances which inhibit enzyme activity and thereby reduce odor formation include trialkyl citrates, such as trimethyl citrate, tripropyl citrate, triisopropyl citrate, tributyl citrate and, in particular, triethyl citrate (Hydagen® C.A.T., Cognis GmbH, Düsseldorf/FRG). The free acid is probably released through the cleavage of the citric acid ester, reducing the pH value of the skin to such an extent that the enzymes are inactivated by acylation. Other substances suitable as esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester and zinc glycinate.

The compositions according to the invention may contain the esterase inhibitors in quantities of 0.01 to 20% by weight, preferably in quantities of 0.1 to 10% by weight and more particularly in quantities of 0.5 to 5% by weight, based on the composition.

Bactericidal or Bacteriostatic Components

Typical examples of suitable bactericidal or bacteriostatic components are chitosan and phenoxyethanol. 5-Chloro-2-(2,4-dichloro-phenoxy)-phenol, which is marketed under the name of Irgasan® by Ciba-Geigy of Basel, Switzerland, has also proved to be particularly effective.

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers. Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chloro-phenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

The compositions according to the invention may contain the bactericidal/bacteriostatic or germ-inhibiting components in quantities of 0.01 to 10% by weight, preferably 0.1 to 5% by weight and more particularly 0.5 to 2% by weight.

Suitable odor absorbers are substances which are capable of absorbing and largely retaining the odor-forming compounds, but are not active against bacteria. They reduce the partial pressure of the individual components and thus also reduce the rate at which they spread. Odor absorbers should not affect the perfume note of a perfume. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, lemon balm oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Perspiration-Absorbing Substances

Suitable perspiration-absorbing substances are modified starch such as, for example, Dry Flo® Plus (National Starch), silicates, talcum, bentonite, montmorillonite, hectorite and other substances of similar modification which appear suitable for absorbing perspiration.

The compositions according to the invention may contain the perspiration-absorbing substances in quantities of 0.1 to 30% by weight, preferably in quantities of 1 to 20% by weight and more particularly in quantities of 5 to 10% by weight.

Other Optional Ingredients

The antiperspirant compositions according to the invention may contain a number of other auxiliaries and additives such as, for example, emulsifiers/surfactants, pearlizing waxes, thickeners, stabilizers, polymers, biogenic agents, film formers, solubilizers, hydrotropes, preservatives, perfume oils and perfumes in general, dyes, etc. which are listed by way of example in the following.

Emulsifiers

The addition of emulsifiers enables small quantities of water-soluble substances and active components and hydrotropes to be incorporated.

Nonionic emulsifiers are preferred for the purposes of the invention. Nonionic emulsifiers are distinguished by their dermatological compatibility and mildness and by their favorable ecotoxicological properties. Among the nonionic emulsifiers, w/o emulsifiers are preferred and may be used in a total quantities of 0 to 20% by weight, preferably 0.1 to 15% by weight and more particularly 0.1 to 10% by weight, based on the total weight of the composition.

Nonionic Emulsifiers

The group of nonionic emulsifiers includes:
(1) products of the addition of 2 to 50 mol ethylene oxide and/or 0 to 20 mol propylene oxide onto linear fatty alcohols containing 8 to 40 carbon atoms, onto fatty acids containing 12 to 40 carbon atoms and onto alkylphenols containing 8 to 15 carbon atoms in the alkyl group;
(2) C_{12/18} fatty acid monoesters and diesters of products of the addition of 1 to 50 mol ethylene oxide onto glycerol;
(3) glycerol monoesters and diesters and sorbitan monoesters and diesters of saturated and unsaturated fatty acids containing 6 to 22 carbon atoms and ethylene oxide adducts thereof;
(4) alkyl mono- and oligoglycosides containing 8 to 22 carbon atoms in the alkyl group and ethoxylated analogs thereof;
(5) products of the addition of 7 to 60 mol ethylene oxide onto castor oil and/or hydrogenated castor oil;
(6) polyol esters and, in particular, polyglycerol esters such as, for example, polyolpoly-12-hydroxystearate, polyglycerol polyricinoleate, polyglycerol diisostearate or polyglycerol dimerate. Mixtures of compounds from several of these classes are also suitable;
(7) products of the addition of 2 to 15 mol ethylene oxide onto castor oil and/or hydrogenated castor oil;
(8) partial esters based on linear, branched, unsaturated or saturated C₆₋₂₂ fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose) or mixed esters, such as glyceryl stearate citrate and glyceryl stearate lactate for example;
(9) wool wax alcohols;
(10) polysiloxane/polyalkyl polyether copolymer or corresponding derivatives;
(11) mixed esters of pentaerythritol, fatty acids, citric acid and fatty alcohol according to and/or mixed esters of fatty acids containing 6 to 22 carbon atoms, methyl glucose and polyols, preferably glycerol or polyglycerol, and
(12) polyalkylene glycols.

The addition products of ethylene oxide and/or propylene oxide onto fatty alcohols, fatty acids, alkylphenols, glycerol monoesters and diesters and sorbitan monoesters and diesters of fatty acids or onto castor oil are known commercially available products. They are homolog mixtures of which the average degree of alkoxylation corresponds to the ratio between the quantities of ethylene oxide and/or propylene oxide and substrate with which the addition reaction is carried out. These emulsifiers are w/o or o/w emulsifiers, depending on the degree of ethoxylation. C_{12/18} fatty acid monoesters and diesters of addition products of ethylene oxide onto glycerol are known as lipid layer enhancers for cosmetic preparations.

According to the invention, particularly suitable and mild emulsifiers are the polyol poly-12-hydroxystearates and mixtures thereof marketed by Cognis Deutschland GmbH under the name of “Dehymuls® PGPH” (w/o emulsifier) or “Eumulgin® VL 75” (mixture with Coco Glucosides in a ratio by weight of 1:1, o/w emulsifier) or “Dehymuls® SBL” (w/o emulsifier). The polyol component of these emulsifiers may be derived from substances which contain at least two, preferably 3 to 12 and more particularly 3 to 8 hydroxyl groups and 2 to 12 carbon atoms.

In principle, suitable lipophilic w/o emulsifiers are emulsifiers with an HLB value of 1 to 8 which are listed in numerous Tables and are well-known to the expert. Some of these emulsifiers are listed, for example, in Kirk-Othmer, “Encyclopedia of Chemical Technology”, 3rd Edition, 1979, Vol. 8, page 913. The HLB value for ethoxylated products may also be calculated to the following formula: HLB=(100-L): 5, where L is the percentage by weight of lipophilic groups, i.e. fatty alkyl or fatty acyl groups, in percent by weight in the ethylene oxide adducts.

Of particular advantage from the group of w/o emulsifiers are partial esters of polyols, more particularly C₃₋₆ polyols, such as for example glyceryl monoesters, partial esters of pentaerythritol or sugar esters, for example sucrose distearate, sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxy-stearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable emulsifiers.

In cases where water-soluble active components and/or small quantities of water are incorporated, it can also be of advantage additionally to use at least one emulsifier from the group of nonionic o/w emulsifiers (HLB value: 8-18) and/or solubilizers. Examples of such emulsifiers are the ethylene oxide adducts mentioned at the beginning with a correspondingly high degree of ethoxylation, for example 10-20 ethylene oxide units for o/w emulsifiers and 20-40 ethylene oxide units for so-called solubilizers. Particularly advantageous o/w emulsifiers for the purposes of the invention are Ceteareth-12 and PEG-20 Stearate. Particularly suitable solubilizers are Eumulgin® HRE 40 (INCI name: PEG40 Hydrogenated Castor oil), Eumulgin® HRE 60 (INCI name: PEG-60 Hydrogenated Castor Oil), Eumulgin® L (INCI name: PPG-1-PEG-9 Laurylglycolether) and Eumulgin® SML 20 (INCI name: Polysorbat-20).

Nonionic emulsifiers from the group of alkyl oligoglycosides are particularly compatible with the skin. C₈₋₂₂ alkyl mono- and oligoglycosides, their production and their use are known from the prior art. They are produced in particular by reacting glucose or oligosaccharides with primary alcohols containing 8 to 22 carbon atoms. So far as the glycoside component is concerned, both monoglycosides where a cyclic sugar unit is attached to the fatty alcohol by a glycoside bond and oligomeric glycosides with a degree of oligomerization of preferably up to about 8 are suitable. The degree of oligomerization is a statistical mean value on which a homolog distribution typical of such technical products is based. Products available under the name of Plantacare® contain a C₈₋₁₆ alkyl group attached by a glucosidic bond to an oligoglucoside unit with an average degree of oligomerization of 1 to 2. The acyl glucamides derived from glucamine are also suitable nonionic emulsifiers. The product marketed under the name of Emulgade® PL 68/50 by Cognis Deutschland GmbH, which is a 1:1 mixture of alkyl polyglucosides and fatty alcohols, is preferred for the purposes of the invention. According to the invention, the mixture of Lauryl Glucoside, Polyglyceryl-2-Dipolyhydroxystearate, glycerol and water which is marketed as Eumulgin® VL 75 may also be used with advantage in accordance with the invention.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Suitable other thickeners are, for example, Aerosil® types (hydrophilic silicas), polysaccharides, more especially xanthan gum, guar-guar, agar-agar, alginates and tyloses, carboxymethyl cellulose and hydroxyethyl and hydroxypropyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable thickeners are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Metal salts of fatty acids such as, for example, magnesium, aluminium and/or zinc stearate or ricinoleate may be used as stabilizers.

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grunau), quaternized wheat poly-peptides, polyethyleneimine, cationic silicone polymers such as, for example, amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacryl-amide/methyl methacrylate/tert.-butylaminoethyl methacrylate/2-hydroxy-propyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

The preparations according to the invention may contain a number of antioxidants which perform a protective function. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmol to μmol/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

In the context of the invention, biogenic agents which may be present in the preparations according to the invention are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract, bambara nut extract, and vitamin complexes.

Typical film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.

Suitable dyes are any of the substances suitable and approved for cosmetic purposes. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS(C.I. 69800) and madder lake (C.I. 58000). Luminol may also be present as a luminescent dye. These dyes are normally used in concentrations of 0.001 to 0.1% by weight, based on the mixture as a whole.

### EXAMPLES

TABLE 1
Antiperspirant formulations: quantities in % by weight of the commercially
available substance, based on the composition as a whole

| Ingredients/INCI | V1 | V2 | V3 | 1 | 2 | 3 | 4 | 5 | 6 |
|---|---|---|---|---|---|---|---|---|---|
| Lanette ® 18 | 20.0 | 16.0 | — | — | | 4.0 | — | — | — |
| Cutina ® HR | — | 4.0 | — | — | 4.0 | 2.0 | — | — | 4.0 |
| Edenor ® OSSG | — | — | 12.0 | — | — | — | 10.0 | 8.0 | — |
| Gelatinization agent GP1 ® | — | — | — | — | — | — | — | 2.0 | |
| Dioctadecylcarbonat | — | — | — | 20.0 | 16.0 | 8.0 | 4.0 | 4.0 | 16.0 |
| Dow Corning ® DC 245 | 33.0 | 33.0 | 37.0 | 33.0 | 33.0 | 35.0 | 35.0 | 35.0 | 33.0 |
| Cetiol ® OE | 9.0 | 9.0 | 10.0 | 9.0 | 9.0 | 10.0 | 10.0 | 10.0 | 9.0 |
| Cetiol ® S | 15.0 | 15.0 | 18.0 | 15.0 | 15.0 | 18.0 | 18.0 | 18.0 | 15.0 |
| Panthenol | | | | | | | | | |
| Rezal ® 36 GP | 23.0 | 23.0 | 23.0 | 23.0 | 23.0 | — | 23.0 | 23.0 | 23.0 |
| Locron ® P | — | — | — | — | — | 23.0 | — | — | — |
| Evaluation | | | | | | | | | |
| Penetration depth [mm] | 4.2 | 4.2 | 4.6 | 4.9 | 4.0 | 8.1 | 4.4 | 4.2 | 4.3 |
| Structure | 3 | 3 | 2 | 2 | 1 | 1 | 1 | 1 | 1 |
| Stability | 4 | 1 | 1 | 1 | 1 | 1 | 1 | 1 | 1 |
| White residue | 2 | 2 | 2 | 2 | 1 | 1 | 1 | 1 | 1 |
| Sensory impression | 2 | 2 | 2 | 1 | 1 | 1 | 1 | 1 | 1 |

Depth of Penetration:

Penetrometer PNR 10 Petrotest (Petrotest Instruments GmbH & Co. KG); microcone: 5.0 g; drop bar: 47.5 g; measuring temperature: 23° C.; penetration time: 5 seconds.

Sensory Evaluation:

The sensory evaluation of the formulations is based on a scale of 1 (very good) to 5 (unsatisfactory). The structure was visually evaluated for particle fineness and smoothness. Stability was evaluated by storage tests over a period of 12 weeks at temperatures of −5° C. to 45° C. White residue was visually determined by application to colorless films and the skin of the forearm.

Antiperspirant formulations 1 to 6 according to the invention based on the long-chain dialkyl carbonates show distinctly better property profiles than Comparison Examples C1 to C3.

### Appendix

1) Cetiol® OE
   INCI: Dicaprylyl Ether
   Manufacturer: Cognis Deutschland GmbH
2) Cetiol® S
   INCI: Diethylhexylcyclohexane
   Manufacturer: Cognis Deutschland GmbH
3) Cutina® HR
   INCI: Hydrogenated Castor Oil
   Manufacturer: Cognis Deutschland GmbH
4) Dow Corning® 245
   INCI: Cyclomethicone
   Manufacturer: Dow Corning
5) Edenor® OSSG
   INCI: 12-Hydroxystearic Acid
   Manufacturer: Cognis Deutschland GmbH
6) Gellant® GP1
   INCI: Dibutyl Lauroyl Glutamide
   Manufacturer: Ajinomoto
7) Lanette® 18
   INCI: Stearyl Alcohol
   Manufacturer: Cognis Deutschland GmbH
8) Locron® P
   INCI: Aluminium Chlorohydrate
   Manufacturer: Clariant
9) Rezal® 36 GP
   INCI: Aluminium Zirconium Tetrachlorohydrex GLY
   Manufacturer: Reheis

1-12. (canceled)

13. An antiperspirant composition comprising:
(a) at least one dialkyl carbonate corresponding to formula I: 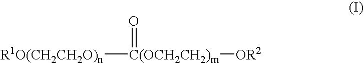
wherein R¹ and R² represent a linear or branched, alkyl and/or alkenyl, group containing from about 16 to 60 carbon atoms, and n and m, independently of one another, represent a number up to 100;
(b) at least one antiperspirant component;
(c) optionally, a wax component other than the dialkyl carbonate; and
(d) optionally, an oil component, and wherein the composition contains less than about 5% by weight, based on the weight of the composition, of water.

14. The composition of claim 13 wherein the composition contains less than about 2% by weight of water.

15. The composition of claim 13 wherein the dialkyl carbonate is present in the composition in an amount of from about 0.1 to 40% by weight, based on the weight of the composition.

16. The composition of claim 13 wherein the dialkyl carbonate is present in the composition in an amount of from about 1 to 30% by weight, based on the weight of the composition.

17. The composition of claim 13 wherein the antiperspirant component is present in the composition in an amount of from about 5 to 30% by weight, based on the weight of the composition.

18. The composition of claim 13 wherein the dialkyl carbonate is dioctadecyl carbonate.

19. The composition of claim 13 wherein the composition has a depth of penetration of from about 1.0 to 40.0 mm after 5 seconds at 23° C.

20. The composition of claim 13 comprising:
(a) from about 1 to 40% by weight of the dialkyl carbonate;
(b) from about 5 to 45% by weight of the antiperspirant component;
(c) from about 0.1 to 20% by weight of a dialkyl ether and/or dialkyl carbonate, other than the dialkyl carbonate of formula I; and
(d) from about 10 to 60% by weight of a volatile silicone oil, all weights being based on the weight of the composition.

21. A process for treating human skin comprising contacting the skin with a composition containing:
(a) at least one dialkyl carbonate corresponding to formula I: 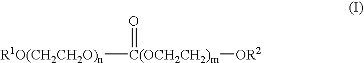
wherein R¹ and R² represent a linear or branched, alkyl and/or alkenyl, group containing from about 16 to 60 carbon atoms, and n and m, independently of one another, represent a number up to 100;
(b) at least one antiperspirant component;
(c) optionally, a wax component other than the dialkyl carbonate; and
(d) optionally, an oil component, and wherein the composition contains less than about 5% by weight, based on the weight of the composition, of water.

22. The process of claim 21 wherein the composition contains less than about 2% by weight of water.

23. The process of claim 21 wherein the dialkyl carbonate is present in the composition in an amount of from about 0.1 to 40% by weight, based +on the weight of the composition.

24. The process of claim 21 wherein the dialkyl carbonate is present in the composition in an amount of from about 1 to 30% by weight, based on the weight of the composition.

25. The process of claim 21 wherein the antiperspirant component is present in the composition in an amount of from about 5 to 30% by weight, based on the weight of the composition.

26. The process of claim 21 wherein the dialkyl carbonate is dioctadecyl carbonate.

27. The process of claim 21 wherein the composition has a depth of penetration of from about 1.0 to 40.0 mm after 5 seconds at 23° C.

28. The process of claim 21 wherein the composition contains:
(a) from about 1 to 40% by weight of the dialkyl carbonate;
(b) from about 5 to 45% by weight of the antiperspirant component;
(c) from about 0.1 to 20% by weight of a dialkyl ether and/or dialkyl carbonate, other than the dialkyl carbonate of formula I; and
(d) from about 10 to 60% by weight of a volatile silicone oil, all weights being based on the weight of the composition.
